(19) United States
(12) Patent Application Publication
(10) Pub. No.: US 2005/0016527 A1
(43) Pub. Date: Jan. 27, 2005

(54) Method and apparatus for testing a metered dose inhaler unit

(76) Inventors: Lee Allen Barger, Durham, NC (US); David Brian Moody, Durham, NC (US); Robert David Schultz, Durham, NC (US); Jonathan Wallace Spencer, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/499,309

(22) PCT Filed: Dec. 17, 2002
(86) PCT No.: PCT/US02/40188

Related U.S. Application Data
(60) Provisional application No. 60/345,120, filed on Dec. 21, 2001.

(51) Int. Cl. A61M 11/00

(52) U.S. Cl. 128/200.23

(57) ABSTRACT

A testing apparatus and method for characterizing the aerodynamic particle size of an atomized medicament emitted from a metered dose inhaler unit during testing with a particle characterization device in which the testing apparatus includes a first clamp of a first fixture in removable clamping and locking engagement with a throat, which is in fluid communication with a particle characterization device, a second clamp in removable clamping and locking engagement with a delivery device of the metered dose inhaler unit having a canister, containing atomized medicament, operatively loaded therein, an alignment mechanism that removably couples the first fixture to the second fixture at an aligned position therefore causing the throat to be in fluid communication with the delivery device, and an actuation assembly mounted to the first fixture having means that operatively thrusts the canister into the delivery device of the metered dose inhaler unit, thus releasing atomized medicament.

### TECHNICAL FIELD

The present invention is generally directed to an apparatus and method for testing a metered dose inhaler unit, and more particularly, to an apparatus and method for aligning the inhaler unit with a particle characterization device and for actuating the inhaler unit in a controlled, automated or manual manner.

### BACKGROUND ART

Medicinal drug formulations useful in inhalation therapy for treating respiratory disorders, such as asthma, are frequently administered to patients orally or nasally in metered, aerosolized doses. Such aerosolized drug formulations are commonly provided in the form of a suspension or emulsion consisting of a pharmaceutical compound in micronized powder form and a suspending medium such as a liquefied gas or propellant. The suspension is initially stored within a sealed canister capable of withstanding the pressure required to maintain the propellant as a liquid. The suspension can then be dispensed in metered, aerosolized doses from the canister upon actuation of an atomized material delivery device such as a metered dose inhaler (MDI) unit. A variety of atomized material devices are well-known in the pharmaceutical industry.

Referring to FIG. 1, an example of a metered dose inhaler (MDI) unit, generally designated 5, is illustrated. MDI unit 5 includes a canister, generally designated 10, operatively loaded into a delivery device, generally designated 22. Canister 10 includes an exposed end 12, a valve end 14 opposing exposed end 12, and an aerosol dose metering valve assembly 16 housed within valve end 14 of canister 10. Valve assembly 16 has a spring-loaded hollow valve stem 18. The details of a typical valve assembly 16 and its interaction with valve stem 18 are well-understood and therefore not specifically shown in FIG. 1. Typically, a suspension or solution (the term “suspension”, as used hereinafter, is taken to mean suspension or solution), and particularly a medicament-containing suspension, is dispensed by actuation of the valve assembly 16. Actuation is accomplished by activation of valve stem 18 through which a metered and substantially repeatable dose of medicament 20 is delivered from canister 10.

In order to deliver medicament 20 to the patient with suitable efficacy, canister 10 preferably operates in conjunction with delivery device 22. Delivery device 22 includes a housing 24 having a first open end 26, a canister cavity 28 into which canister 10 is operatively loaded, and a mouthpiece 30 having a second open end 32. A nozzle assembly 34 is disposed within housing 24 of delivery device 22. Nozzle assembly 34 includes a valve stem receiving bore 36 fluidly communicating with a nozzle orifice 38. Nozzle orifice 38 is aimed toward second open end 32 of mouthpiece 30.

In order to receive a properly metered dose of medicament 20 from canister 10, the patient loads canister 10 into canister cavity 28 of delivery device 22 through first open end 26 of delivery device 22 until valve stem 18 of valve assembly 16 is fitted into valve stem receiving bore 36 of nozzle assembly 34. With canister 10 so installed, exposed end 12 of canister 10 typically extends to some degree outside of housing 24 of delivery device 22. The patient then aims mouthpiece 30 towards, or places mouthpiece 30 into, his or her mouth and pushes with a downward force on exposed end 12 of canister 10. As a result, canister 10 is displaced in a downward manner, with respect to valve stem 18, thereby actuating valve assembly 16 and causing a metered dose of medicament 20 to be emitted from valve stem 18, through nozzle orifice 38, and through second open end 32 of mouthpiece 30.

As the suspension is forced from canister 10 through valve stem 18 by the high vapor pressure of the liquid propellant, the propellant rapidly vaporizes (i.e., boils), therefore leaving a fast moving stream of atomized particles of medicament 20. This atomized medicament 20 is directed into the mouth of the patient by mouthpiece 30 of delivery device 22, which has previously been aimed toward, or inserted into, the patient's mouth. Owing to the design of valve assembly 16, the design of nozzle assembly 34, and the pressure differential between the interior of canister 10 and the exterior ambient atmosphere, a short burst of precisely metered, atomized medicament 20 is thereby delivered to the patient upon actuation. The patient simultaneously inhales medicament 20 into his or her lungs concurrently with actuation of valve assembly 16.

Patients frequently rely upon medication delivered by MDI units 5 for rapid or sustained treatment of respiratory disorders, such as asthma, which are debilitating and in some instances even life threatening. As a result, it is essential that the prescribed dose of aerosolized drug formulation being delivered to the patient's lungs consistently meet the specifications claimed by the manufacturer and comply with the stringent requirements set forth by drug regulatory authorities, such as the Food and Drug Administration (FDA). Therefore, testing of MDI units 5 for effective and efficient drug delivery by valve assembly 16 is a necessary part of a manufacturer's quality assurance procedure.

One aspect of the testing of MDI units 5 for effective and efficient drug delivery involves measuring or characterizing the aerodynamic particle size of atomized medicament 20, which is released upon actuation of valve assembly 16 that is disposed within valve end 14 of canister 10 containing the suspension. If the aerodynamic particle size of atomized medicament 20 is too large, medicament 20 will be deposited on the inside surface of the patient's throat. As a result, the prescribed dose of respiratory medicament 20 is prevented from reaching the patient's lungs, thereby thwarting the patient from receiving the benefit of the potentially life saving therapeutic effects of medicament 20. If, on the other hand, the aerodynamic particle size of atomized medicament 20 is too small, medicament 20 will not be adequately transferred to the patient's lungs. As a result, the prescribed dose of respiratory medicament 20 is simply exhaled by the patient without having exhibited any substantial therapeutic effect.

A particle characterization device is an instrument often utilized for performing analytical assays on the aerodynamic particle size of atomized medicament 20 released by MDI units such as MDI unit 5, in determining the effectiveness and efficiency of drug delivery. Referring to FIG. 2, one specific type of particle characterization device particularly suited for aerodynamically sizing atomized medicament 20 within liquid aerosols is a particle size impactor, such as a multi-stage cascade impactor, generally designated 42. Cascade impactor 42 is an eight stage cascade impactor, which is available from Andersen Instruments, Inc. Cascade impactor 42 relies upon inertial impaction to characterize aerodynamic particle size of atomized medicament 20 released by the MDI unit 5. More specifically, cascade impactor 42 is constructed of a number (typically about eight) of classification stages, generally designated ST₀-ST₇ in FIG. 2. Classification stages ST₀-ST₇ enable classification of aerodynamic particle size of atomized medicament 20, which typically ranges from about 9 micrometers (μm) to about 0.4 μm at approximately 28.3 Ipm. As shown in FIG. 2, cascade impactor 42, includes a classification stage zero ST₀, classification stages ST₁-ST₇, a final filter 44, a base element 46, and an inlet cone 48. Each classification stage ST₀-ST₇ includes a jet plate and a stainless steel impaction disc or filtration media substrate disposed adjacent to the jet plate (not specifically shown). The jet plate has a plurality of jet orifices through which a jet stream passes at a given velocity. The impaction disc has an impaction surface. Final filter 44 collects all particles smaller than 0.4 μm. Inlet cone 48 allows the cascade impactor 42 to operate at higher flow rates so as to enable collection of submicron sized particulates.

Referring to FIGS. 3 and 4, a hollow throat, generally designated 58, is often utilized to enhance or modify particulate flow conditions into cascade impactor 42 and, in the present case, can be utilized to provide fluid communication between MDI unit 5 and cascade impactor 42 during testing of aerosolized drug formulations. Throat 58 includes a tapered end 60 and a flanged neck end 62. As shown in FIG. 4, tapered end 60 of throat 58 is inserted into inlet cone 48 of cascade impactor 42.

During testing, a stream of atomized particles under investigation is conducted through throat 58 into cascade impactor 42. Upon entering cascade impactor 42, the stream of atomized medicament becomes entrained within the jet stream passing through the jet orifices of the jet plates and around the impaction discs. At each classification stage ST₀-ST₇, entrained atomized medicament 20 having a large aerodynamic particle size with enough inertia settle upon the impaction surface of the impaction disc associated with that particular classification stage ST. On the other hand, entrained particles of atomized medicament 20 having a small aerodynamic particle size remain entrained within the jet stream and are later deposited upon the impaction surface of an impaction disc present within a subsequent classification stage ST.

Higher jet velocities enable smaller particles to be characterized more efficiently. The aerodynamic particle size of collected atomized medicament 20 depends upon the velocity of the jet stream passing through the jet orifices within each classification stage ST₀-ST₇, the distance between the jet orifices of the jet plate and the impaction surface of the impaction disc, and the collection characteristics of the preceding classification stage ST. Cascade impactor 42 is versatile in that all aerosolized drug formulations can be classified by extensive experimental proofs and empirical verification. Furthermore, once certain properties of the collected particles of atomized medicament 20 are determined, cascade impactor 42 enables comprehensive aerosolized drug formulation definition.

One problem encountered when utilizing cascade impactor 42 during characterization of the aerodynamic particle size of atomized medicament 20, which has been released by an atomized material delivery device such as MDI unit 5, is ensuring that an efficient and effective fluid communication is established between MDI unit 5 and cascade impactor 42. Therefore, providing for a proper alignment of throat 58, which is connected to inlet cone 48 of cascade impactor 42, with mouthpiece 30 of delivery device 22 of MDI unit 5 would aid in improving fluid communication between MDI unit 5 and cascade impactor 42. As a result, deposition of medicament 20 on the inside surface of throat 58 would be reduced, thus providing for the attainment of more accurate and reliable test results.

Another problem encountered during characterization of the aerodynamic particle size of atomized medicament 20 is that cascade impaction testing procedures are inherently variable due to the degree of human involvement or interaction typically required. In particular, it has been observed that the amount of force manually applied to canister 10 during actuation of MDI unit 5 by a patient, and the length of time during which this actuation force is applied, can significantly determine whether a therapeutically effective dose of medicament is successfully delivered to the patient's lungs. This variability likewise attends the manual testing procedures performed by a researcher while using an atomized material delivery device such as MDI unit 5 to inject a particulate stream into cascade impactor 42.

The present invention is provided to address these and other problems associated with the testing of particulates such as atomized medicament formulations.

Therefore, in an effort to eliminate the testing variability attributable to various human analytical techniques, it is now proposed herein that the flow conditions from MDI unit 5 to cascade impactor 42 can be improved through the use of an alignment mechanism to enhance the coupling between MDI unit 5 to cascade impactor 42. It is further proposed that the magnitude of the downward force typically exerted against exposed end 12 of canister 10 by a laboratory technician during a quality assurance or other testing procedure can instead be applied by a mechanical or electromechanical means, such as some type of a linear actuator, which simulates the downward force exerted by a patient during normal use of MDI unit 5. In addition, it has now been found that the time of actuation (that is, the period during which the downward force is exerted against exposed end 12 of canister 10) can be repeatably controlled, and deliberately varied for assaying purposes, through application of a mechanical or electromechanical means, as more fully disclosed hereinbelow.

### DISCLOSURE OF THE INVENTION

The present invention generally provides a testing apparatus and method for alignment and automated actuation during characterization of the aerodynamic particle size of an atomized medicament, which preferably has been emitted from a metered dose inhaler (MDI) unit, conducted through a hollow throat, and injected into a particle size impactor.

According to one embodiment of the present invention, the testing apparatus includes a first fixture having a first clamp that is adjustable between an open position and a closed clamping position, a second fixture having a second clamp that is adjustable between an open position and a closed clamping position, an alignment mechanism that removably couples the first fixture to the second fixture at an aligned position of the testing apparatus, and an actuation assembly mounted to the first fixture and which includes a reciprocative member.

Preferably, the first clamp is adjustable about a first hinge between the open position and the closed clamping position of the first clamp.

Preferably, the first clamp includes a first locking mechanism. More preferably, the first locking mechanism is pivotable about a first pivot pin disposed within the first clamp between a disengaged position and an engaged position. It is also preferable that the first locking mechanism include a rotatable first locking element that is engageable with the first clamp between a loosened position and a tightened position.

In one form of this embodiment, the first clamp removably engages a throat.

In another form of this embodiment, the first clamp removably engages an atomized material delivery device. Preferably, the atomized material delivery device is a metered dose inhaler unit.

Preferably, the second clamp is adjustable about a second hinge between the open position and the closed clamping position of the second clamp.

Preferably, the second clamp includes a second locking mechanism. More preferably, the second locking mechanism is pivotable about a second pivot pin disposed within the second clamp between a disengaged position and an engaged position. It is also preferable that the second locking mechanism include a rotatable second locking element that is engageable with the second clamp between a loosened position and a tightened position.

In one form of this embodiment, the second clamp removably engages an atomized material delivery device. According to this aspect, it is preferable that the atomized material delivery device is a metered dose inhaler unit.

In another form of this embodiment, the second clamp removably engages a throat.

Preferably, the alignment mechanism includes an alignment post protruding from the first clamp and an alignment bore formed within the second clamp. More preferably, the alignment post is engagingly inserted into the alignment bore at an aligned position of the testing apparatus.

According to an alternative aspect of the invention, the alignment mechanism includes an alignment post protruding from the second clamp and an alignment bore formed within the first clamp. According to this aspect of the invention, it is preferable that the alignment post be engagingly inserted into the alignment bore at an aligned position of the testing apparatus.

Preferably, when the testing apparatus is in its aligned position, a throat is in fluid communication with an atomized material delivery device. More preferably, at the aligned position of the testing apparatus, the throat is mounted to the first fixture at the closed clamping position of the first clamp, and the atomized material delivery device is mounted to the second fixture at the closed clamping position of the second clamp. At the aligned position of the testing apparatus, it also preferable that the reciprocative member of the actuation assembly be in axial alignment with the atomized material delivery device.

In one embodiment, the actuation assembly includes a pneumatic cylinder disposed in operative communication with the reciprocative member. In one embodiment, the actuation assembly utilizes a stepper motor in operative communication with the reciprocative member.

Within the scope of the invention, the actuation assembly can include alternative mechanisms, such as a solenoid in operative communication with the reciprocative member.

In one embodiment, the actuation assembly includes an axial force measuring transducer. In one embodiment, the actuation assembly includes a displacement measuring transducer to measure acceleration. In one embodiment, the testing apparatus also comprises a data-receiving device that electrically communicates with the above transducers.

Preferably, the testing apparatus comprises a power supply source communicating with the actuation assembly. Preferably, the power supply source is a pressurized air source and the actuation assembly includes a pneumatic cylinder. However, other actuation mechanisms are contemplated within the scope of the invention. Thus, in other embodiments of the invention, the power supply source is an electrical energy source and the actuation assembly includes a solenoid.

Preferably, the testing apparatus comprises a control module for controlling the supply of power to the actuation assembly. More preferably, the control module includes a timer device for controlling the period of time over which the reciprocative member is extended by the actuation assembly.

In at least one embodiment of the invention, the first clamp is adapted for removable clamping engagement with a throat, which is in fluid communication with a particle size impactor such as a multi-stage cascade impactor. The second clamp is adapted for removable clamping engagement with a delivery device, wherein the delivery device has a canister cavity with a canister operatively loaded therein, and containing a suspension of medicament.

According to this embodiment, the alignment mechanism, which removably couples the first fixture to the second fixture at an aligned position, has an alignment bore formed within the second clamp and extends through the second fixture, and an alignment post protruding from the first clamp of the first fixture towards the alignment bore of the second fixture. Once the testing apparatus is in an aligned position, the alignment post of the first fixture is engagingly inserted into the alignment bore of the second fixture. As a result, the first fixture is removably coupled to the second fixture, therefore causing the throat to be in fluid communication with the delivery device of the MDI unit.

The alignment mechanism of the testing apparatus insures that an efficient and effective fluid communication is present between the delivery device of the MDI unit, which has the canister containing atomized medicament operatively loaded into the delivery device, and the throat, which is connected to the cascade impactor. As a result, the testing apparatus enables more consistent deposition of the medicament on the inside surface of the throat, therefore providing for the attainment of more accurate, repeatable, and reliable test results.

The alignment mechanism of the testing apparatus also ensures that the actuation assembly, which includes a reciprocative member, is located in an initial position above the canister and is axially aligned with the canister, when the first fixture is coupled to the second fixture at the aligned position of the testing apparatus. Actuation generally occurs when the reciprocative member of the actuation assembly thrusts the canister into the canister cavity of the delivery device, thus releasing atomized medicament.

The actuation assembly of the testing apparatus eliminates testing variability, which is attributable to various human analytical techniques, by replacing the traditional source (i.e., a laboratory technician) of the downward force exerted against the canister with the reciprocative member, which is preferably an automated pneumatic piston. As a result, the testing apparatus eliminates testing variability during characterization of the aerodynamic particle size of the atomized medicament, which has typically plagued quality assurance testing procedures in the past due to the high degree of human involvement or interaction historically required.

According to another embodiment of the present invention, an apparatus is provided for aligning an atomized material delivery device such as a metered dose delivery unit with a particle characterization device. The apparatus comprises an inlet conduit, a first fixture removably secured to the inlet conduit, an atomized material delivery device having an outlet conduit, a second fixture removably secured to the delivery device, and an alignment mechanism. The alignment mechanism intercouples the first fixture to the second fixture at an aligned position of the apparatus. The inlet conduit fluidly communicates with the outlet conduit at the aligned position of the apparatus.

Preferably, the apparatus comprises a particle characterization component which includes the inlet conduit. More preferably, the particle characterization component is a throat.

Preferably, the apparatus comprises an adapter element that interconnects the inlet conduit with the outlet conduit at the aligned position of the apparatus.

Preferably, the alignment mechanism includes a guide member supported by the first fixture. The guide member is inserted into a bore of the second fixture at the aligned position of the apparatus.

Preferably, the apparatus comprises an actuation mechanism that includes a reciprocative member axially aligned with the delivery device at the aligned position of the apparatus. More preferably, the apparatus comprises an actuation control module disposed in operative communication with the actuation mechanism. It is further preferable that the apparatus comprise an axial force measurement transducer operatively mounted to the actuation mechanism.

According to this embodiment, the first fixture, the second fixture, the alignment mechanism, the actuation mechanism, the actuation control module, and the axial force measurement transducer are provided according to preferred and/or alternative embodiments and forms as described hereinabove.

According to yet another embodiment of the present invention, a testing apparatus is provided for providing alignment and automated actuation during particle analysis of an atomized medicament emitted from a delivery device and conducted into a particle characterization device. The testing apparatus comprises a particle characterization device that includes an inlet conduit, a first fixture, a metered dose inhaler unit, a second fixture, an alignment mechanism, and an actuation assembly. The first fixture includes a fist clamp that is in removable clamping engagement with the inlet conduit. The metered dose inhaler unit includes a delivery device and a particle-containing canister operatively loaded in the delivery device. The second fixture includes a second clamp that is in removable clamping engagement with the delivery device of the metered dose inhaler unit. The alignment mechanism includes an alignment post protruding from the first clamp and an alignment bore formed within the second clamp. The alignment post is adapted to be engagingly inserted into the alignment bore at an aligned position of the testing apparatus, so as to provide fluid communication between inlet conduit and the delivery device of the metered dose inhaler unit. The actuation assembly is mounted to the first fixture, and includes a reciprocative member positioned in operative alignment with the canister. The reciprocative member is adapted to thrust the canister into the delivery device to cause the delivery device to release a metered dose of particles from the canister.

Preferably, the reciprocative member includes an axial force measuring transducer.

Preferably, the testing apparatus comprises a control system for controlling the operation of the actuation assembly.

According to still another embodiment of the present invention, a method is provided for testing the aerodynamic particle size of an atomized medicament. In the method, an inlet portion of a particle characterization device is engaged with a first fixture. A canister containing particles is operatively loaded into an atomized material delivery device. The delivery device is engaged with a second fixture. An aligned position of the testing apparatus is effected by removably coupling the first fixture to the second fixture so as to provide fluid communication between the inlet conduit and the delivery device. The release of particles from the canister is then actuated.

Preferably, the step of effecting the aligned position of the testing apparatus includes aligning an alignment post protruding from the first fixture with an alignment bore formed within the second fixture, and inserting the alignment post into the alignment bore.

Preferably, the step of actuating the release of particles from the canister includes causing a linearly translatable member to impact the canister with an axially oriented force. More preferably, the method comprises the step of measuring a magnitude of an axial force imparted to the canister during the actuating step.

According to another method of the present invention, the step of actuating the release of particles from the canister includes manually applying a force to the canister.

According to an additional aspect of the present invention, a medicament tested using the apparatus and method disclosed herein is provided. The medicament includes a drug component selected from the group consisting of analgesics, anginal preparations, anti-allergenics, anti-infectives, antihistamines, anti-inflammatories, anti-tussives, bronchodilators, adenosine 2a agonists, α₄ integrin inhibitors, diuretics, anticholinergics, hormones, xanthines, therapeutic proteins, therapeutic peptides, vaccines, diagnostics, gene therapies, and salts, esters and solvates thereof, and combinations thereof.

It is therefore an object of the present invention to provide a testing apparatus that enables an efficient and effective fluid communication between the throat and the delivery device of the MDI unit.

It is therefore another object of the present invention to provide a testing apparatus having a reciprocative member positioned in operative alignment with the canister and adapted to thrust the canister into the delivery device to actuate release of a metered dose of atomized medicament.

Some of the objects of the invention having been stated hereinabove, other objects will be evident as the description proceeds, when taken in connection with the accompanying drawings as best described hereinbelow.

### BRIEF DESCRIPTION OF THE DRAWINGS

FIG. 1 is a vertical cross-sectional view of a known metered dose inhaler (MDI) unit with a delivery device having a canister operatively loaded therein, and during operation thereof;

FIG. 2 is a perspective view of a known multi-stage cascade impactor;

FIG. 3 is a perspective view of a known throat adapted for use with the cascade impactor illustrated in FIG. 2;

FIG. 4 is a perspective view of the throat of FIG. 3 connected to the cascade impactor of FIG. 2;

FIG. 5 is a perspective view of a testing apparatus provided in accordance with the present invention;

FIG. 6A is a perspective view of a first clamp of a first fixture provided with the testing apparatus of FIG. 5, wherein the first clamp is shown in an open position as the first clamp is mounted to the throat of FIG. 3;

FIG. 6B is a perspective view of the first clamp of the first fixture in a closed position in removable engagement with the throat of FIG. 3;

FIG. 6C is a side elevation view of the first clamp of the first fixture showing a first hinge thereof;

FIG. 7A is a perspective view of a second clamp of a second fixture provided with the testing apparatus of FIG. 5, wherein the second clamp is shown in an open position as the second clamp is mounted to the delivery device of the MDI unit illustrated in FIG. 1;

FIG. 7B is a perspective view of the second clamp of the second fixture in a closed position in removable engagement with the delivery device of the MDI unit illustrated in FIG. 1;

FIG. 7C is a side elevation view of the second clamp of the second fixture showing a second hinge thereof;

FIG. 8 is a perspective view of the assembled testing apparatus of FIG. 5 at an aligned position in accordance with the present invention;

FIGS. 9A, 9B, and 9C are vertical cross-sectional views of the testing apparatus during operation thereof as the actuator moves from retracted to partially extended to fully extended positions, respectively; and

FIG. 10 is a schematic diagram of a control unit in operative communication with the testing apparatus illustrated in FIGS. 5-9.

FIG. 11 is a perspective view of an assembled testing apparatus of FIG. 5 in accordance with the present invention.

FIG. 12 is a perspective view of an assembled testing apparatus in accordance with the present invention.

FIG. 13 is a cross-sectional view of an assembled testing apparatus in accordance with the present invention.

### DETAILED DESCRIPTION OF THE INVENTION

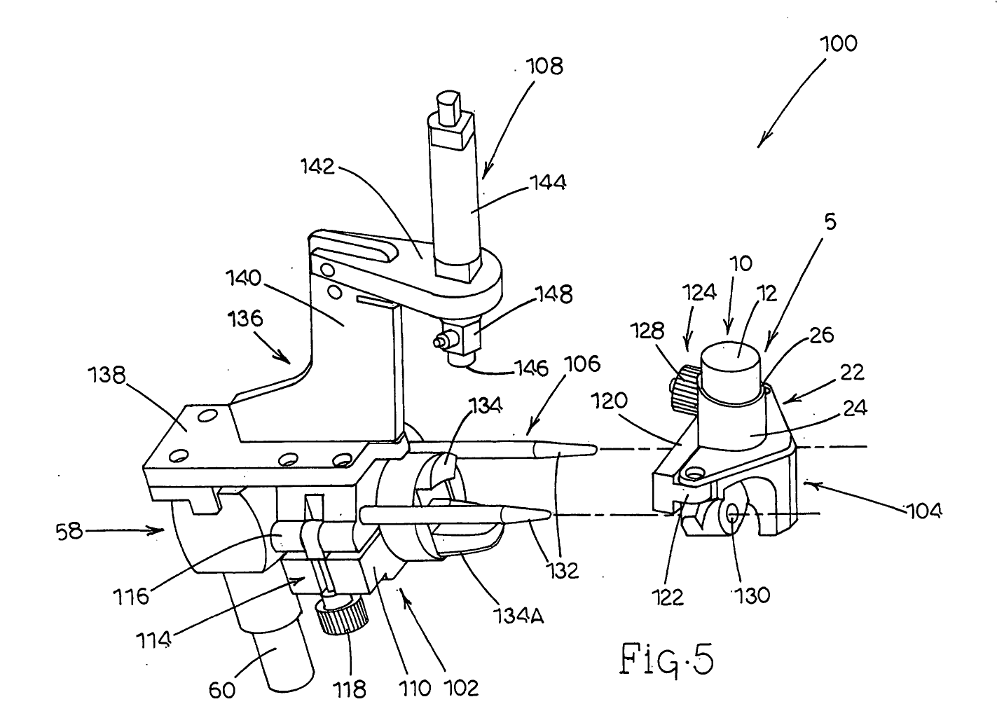

Referring now to FIG. 5, a testing apparatus, generally designated 100, is illustrated in accordance with the present invention. Testing apparatus 100 includes a first fixture, generally designated 102; a second fixture, generally designated 104; an alignment mechanism, generally designated 106, that removably couples first fixture 102 to second fixture 104; and an actuation assembly, generally designated 108, mounted to first fixture 102.

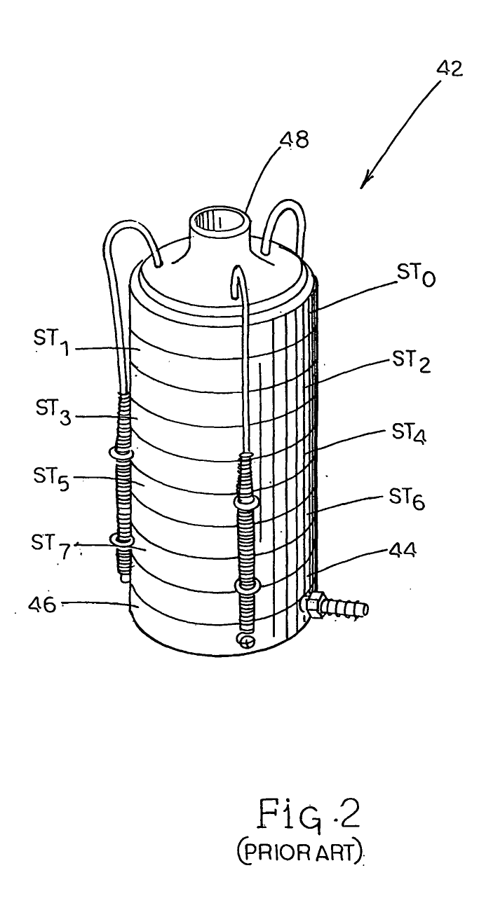

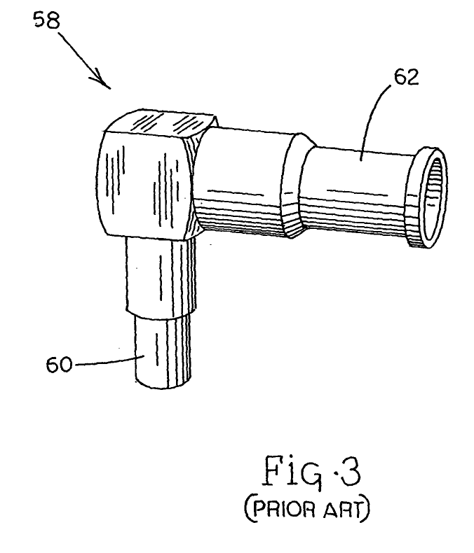

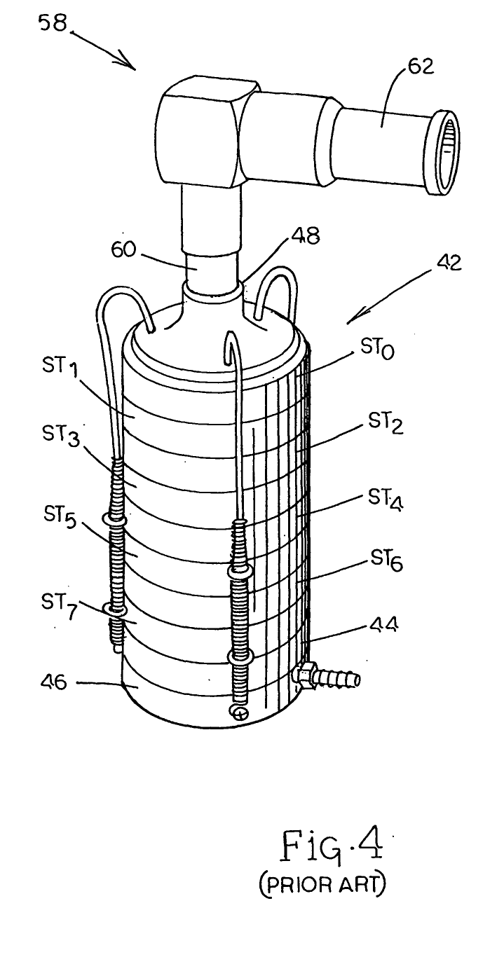

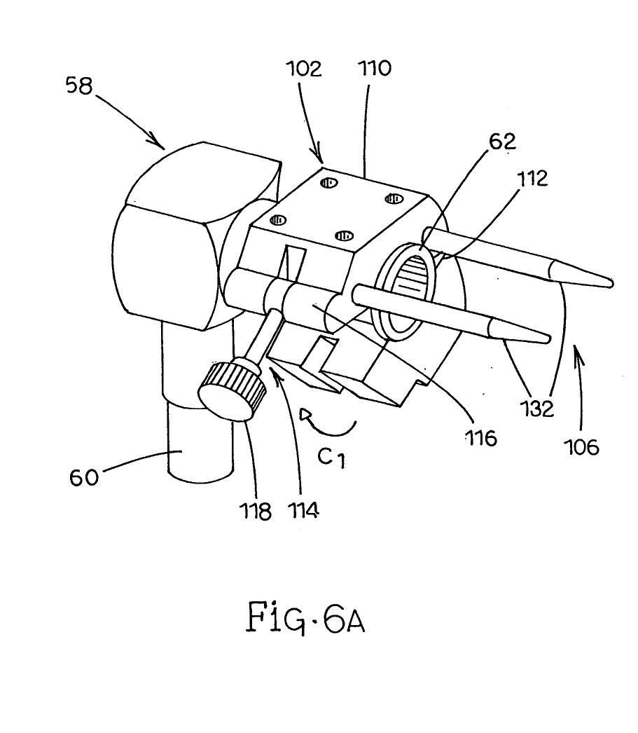

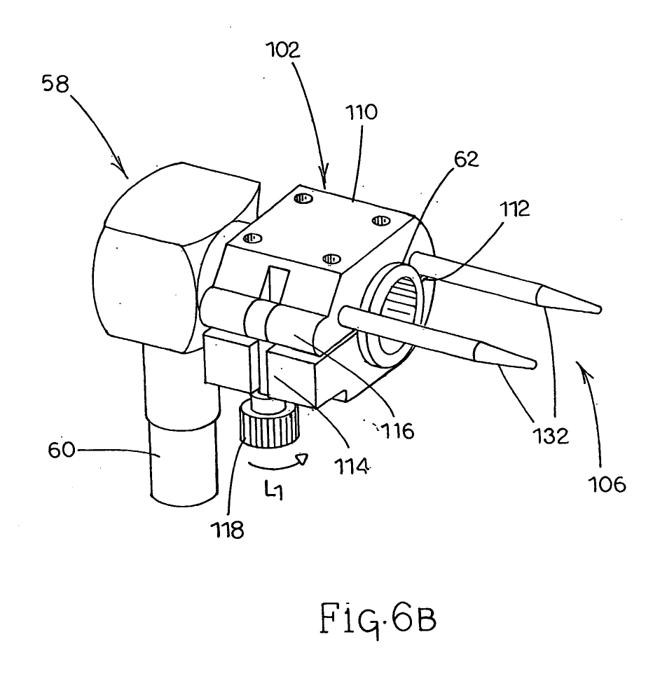

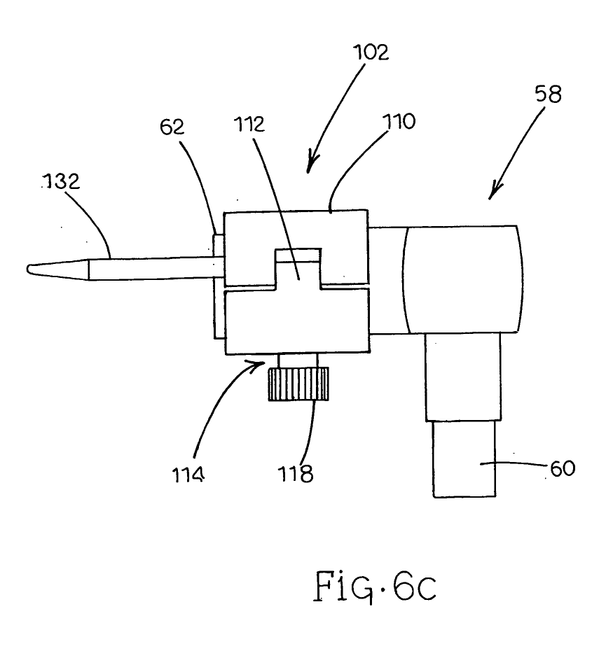

Referring generally to FIGS. 6A, 6B, and 6C, first fixture 102 is illustrated in detail according to one embodiment of the present invention. First fixture 102 generally provides a means for attaching first fixture 102 to an inlet structure (such as throat 58 illustrated in FIGS. 3 and 4) of a particle characterization device (such as cascade impactor 42 illustrated in FIGS. 2 and 4). Accordingly, first fixture 102 includes a first clamp 110. Preferably, first clamp 110 has a pivot axis such as defined by a first hinge 112 and a first locking mechanism, generally designated 114. First clamp 110 is adjustable about first hinge 112 between an open position and a closed clamping position. FIG. 6A is a perspective view of first clamp 110 of first fixture 102 in an open position as first clamp 110 is mounted to throat 58. An arrow, designated C₁ in FIG. 6A, illustrates the closing motion of first clamp 110 about first hinge 112.

FIG. 6B is a perspective view of first clamp 110 of first fixture 102 in a closed clamping position. First locking mechanism 114 is pivotable about a first pivot pin 116 disposed within first clamp 110, between a disengaged position and an engaged position as shown in FIGS. 6A and 6B, respectively. Preferably, first locking mechanism 114 includes a rotatable first locking element 118 threaded thereon. An arrow, designated L₁ in FIG. 6B, illustrates first locking element 118 undergoing a locking motion to secure first locking mechanism 114 in engagement with first clamp 110. First locking element 118 can thus be loosened or tightened against first clamp 110 in order to decrease or enhance the locking engagement between first clamp 110 and throat 58, respectively.

FIG. 6C further illustrates first clamp 110 of first fixture 102 in removable clamping engagement with flanged neck end 62 of throat 58. As shown in FIG. 6C, first clamp 110 of first fixture 102 is in a closed clamping position, with first clamp 110 mounted to throat 58, and first locking element 118 of first locking mechanism 114 is in a tightened position in locking engagement between first clamp 110 and throat 58.

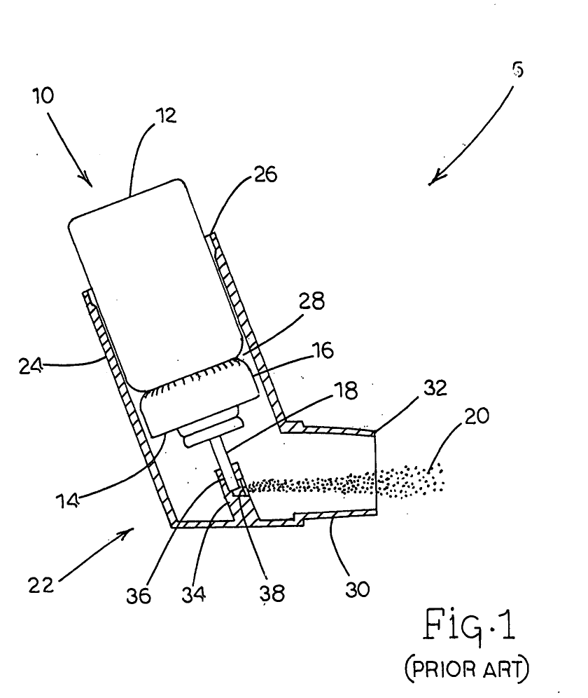

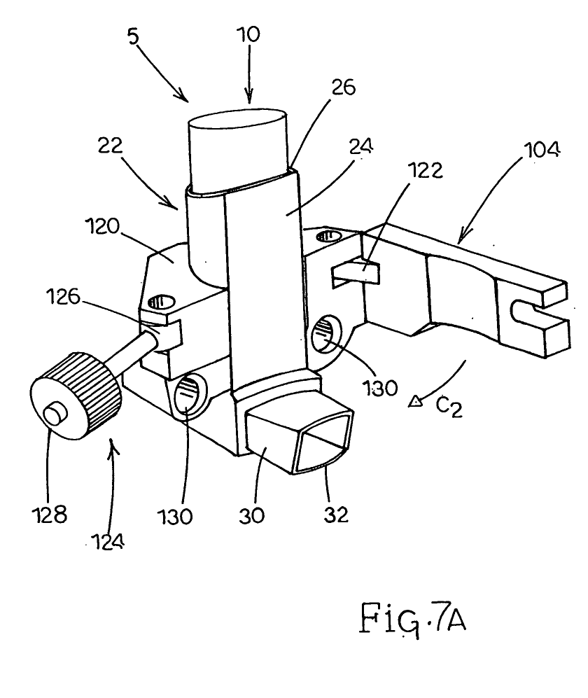

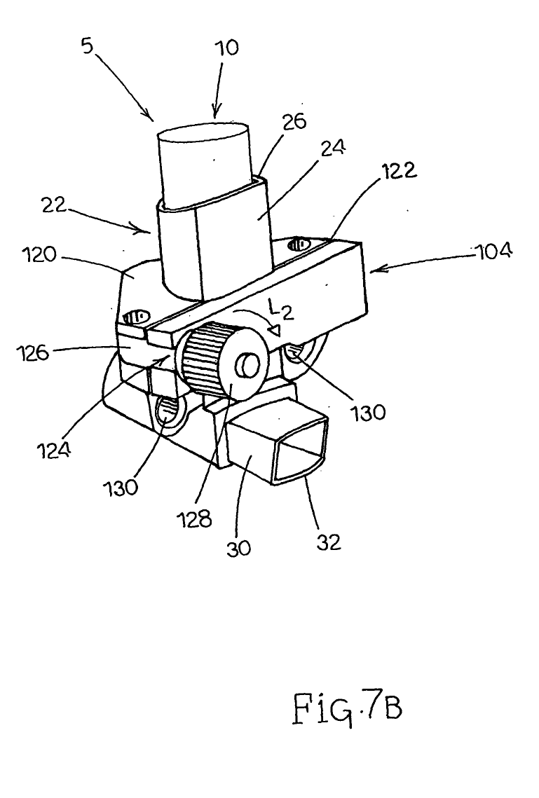

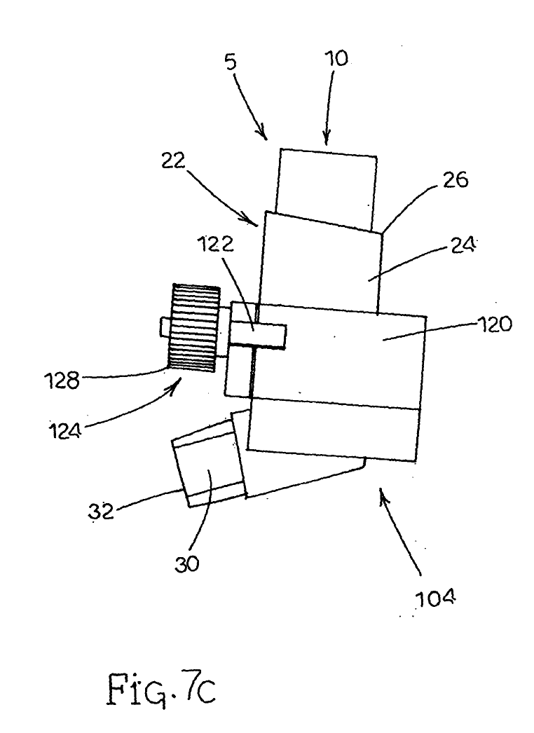

Referring now to FIGS. 7A, 7B, and 7C, second fixture 104 is illustrated according to one embodiment of the present invention. Second fixture 104 generally provides a means for mounting or securing an atomized material delivery device such as MDI unit 5 (an example of which is illustrated in FIG. 1). For this purpose, second fixture 104 includes a second clamp 120. Second clamp 120 has a pivot axis such as defined by a second hinge 122 and a second locking mechanism, generally designated 124. Second clamp 120 is adjustable about second hinge 122 between an open position and a closed clamping position. FIG. 7A is a perspective view of second clamp 120 in an open position. An arrow, designated C₂ in FIG. 7A, illustrates the closing motion of second clamp 120 about second hinge 122.

FIG. 7B is a perspective view of second clamp 120 of second fixture 104 in a closed clamping position. Second locking mechanism 124 is pivotable about a second pivot pin 126 disposed within second clamp 120, between a disengaged position and an engaged position as shown in FIGS. 7A and 7B, respectively. Preferably, second locking mechanism 124 includes a rotatable second locking element 128 threaded thereon. An arrow, designated L₂ in FIG. 7B, illustrates second locking element 128 undergoing a locking motion to secure second locking mechanism 124 in engagement with second clamp 120. Second locking element 128 can thus be loosened or tightened against second clamp 120 in order to decrease or enhance the locking engagement between second clamp 120 and delivery device 22 of MDI unit 5, respectively.

FIG. 7C further illustrates second clamp 120 of second fixture 104 in removable clamping engagement with delivery device 22 of MDI unit 5. As shown in FIG. 7C, second clamp 120 of second fixture 104 is in a closed clamping position, with second clamp 120 mounted to delivery device 22, and second locking element 128 of second locking mechanism 124 is in a tightened position in locking engagement between second clamp 120 and delivery device 22.

As an alternative, second clamp 120 could be constructed as a draw latch that includes a pivoting buckle-type mechanism for engaging with delivery device 22 of MDI unit 5. The use of a draw latch would eliminate the need for a rotatable second locking element 128.

Referring back to FIG. 5, alignment mechanism 106 of testing apparatus 100 removably couples first fixture 102 to second fixture 104 at an aligned position of testing apparatus 100. Alignment mechanism 106 includes one or more alignment bores 130 and one or more corresponding alignment posts 132. In a preferred embodiment, alignment bore 130 is formed within second clamp 120 and extends through second fixture 104, while alignment post 132 protrudes from first clamp 110 of first fixture 102, towards alignment bore 130 of second fixture 104. In an alternative embodiment, alignment bore 130 could be formed within first clamp 110 and extend through first fixture 102, while alignment post 130 could protrude from second clamp 120 of second fixture 104, toward alignment bore 130 of first fixture 102.

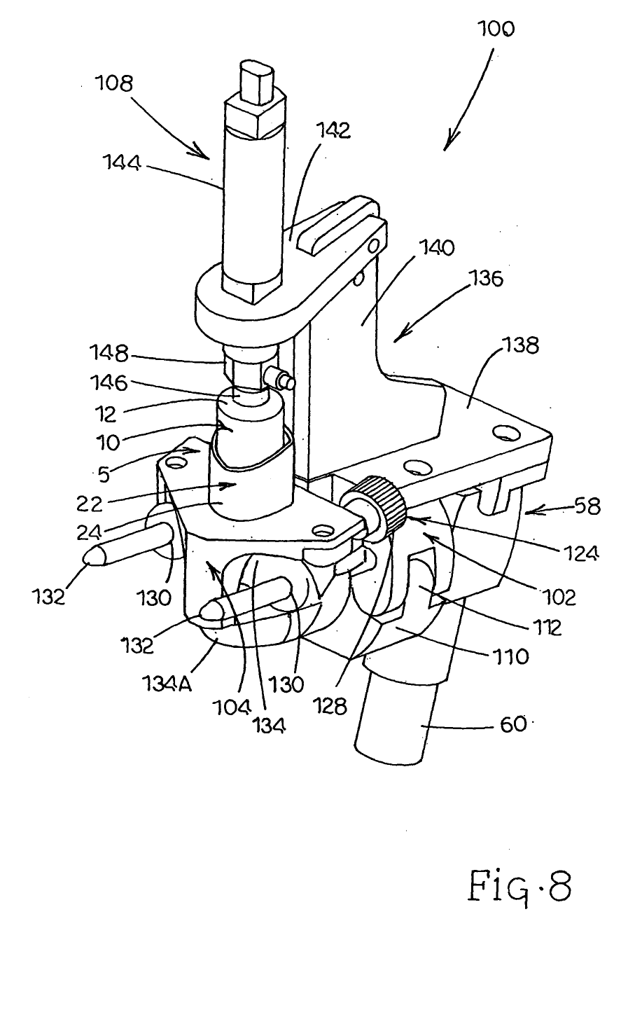

Referring to FIGS. 5 and 8, testing apparatus 100 is assembled by removably coupling first fixture 102 to second fixture 104 with the use of alignment mechanism 106. Accordingly, testing apparatus 100 can be brought into an aligned position by engagingly inserting alignment post 132 of first fixture 102 into alignment bore 130 of second fixture 104. FIG. 8 illustrates the fully aligned position of testing apparatus 100. As shown in FIG. 8, once testing apparatus 100 has been brought into its aligned position in this manner, flanged neck end 62 (see FIGS. 3 and 4) of throat 58 fluidly communicates with mouthpiece 30 of delivery device 22 with enhanced efficiency and consequently improved flow conditions. First fixture 102 may also include a mouthpiece adapter 134 affixed to throat 58 to facilitate the efficient fluid communication between flanged neck end 62 of throat 58 and mouthpiece 30 of delivery device 22. Preferably, mouthpiece adapter 134 is constructed from a resilient material so as to improve the seal at its interface with throat 58.

As illustrated in FIG. 5, mouthpiece adapter 134 is designed so as to provide a substantially non-disruptive transition between the cross-sectional flow area provided by second open end 32 of mouthpiece 30 (see, for example, FIG. 7A) and the often different cross-sectional flow area provided by flanged neck end 62 of throat 58 (see, for example, FIG. 3). Moreover, it can also be seen that mouthpiece adapter 134 Includes a platform 134A to provide additional support for MDI unit 5 when testing assembly 100 is in the aligned position. However, platform 134A is not necessarily required in the invention in view of the support provided by alignment post 132.

Alignment post 132, protruding from first clamp 110 of first fixture 102, has a rounded cross-sectional profile, such as circular, semi-circular, oval, or the like. Therefore, in order to engagingly insert alignment post 132 into alignment bore 130, formed within second clamp 120 of second fixture 104, alignment bore 130 should have a matching rounded cross-sectional profile. Alternatively, alignment post 132 may have a polygonal cross-sectional profile, such as square, rectangular, triangular, pentagonal, hexagonal, or the like. In this latter case, alignment bore 130 should have a matching polygonal cross-sectional profile that corresponds to the particular cross-sectional profile of alignment post 132.

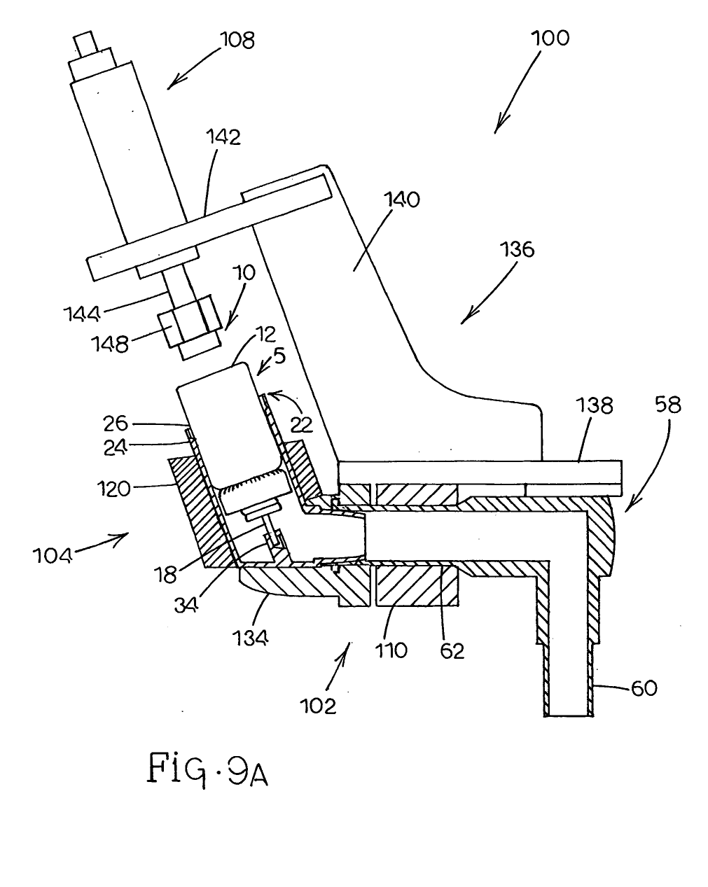

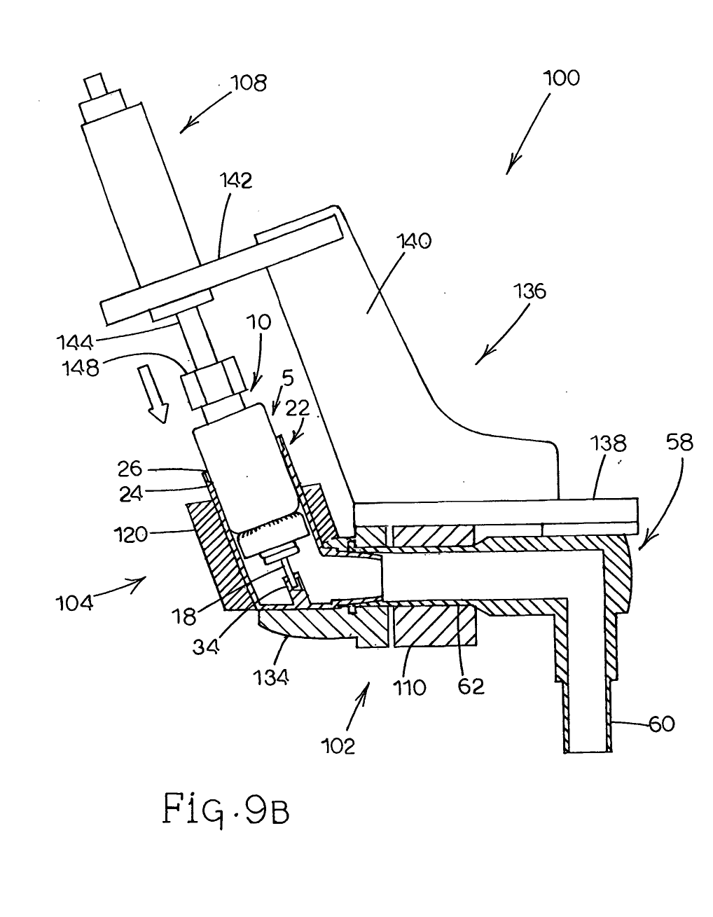

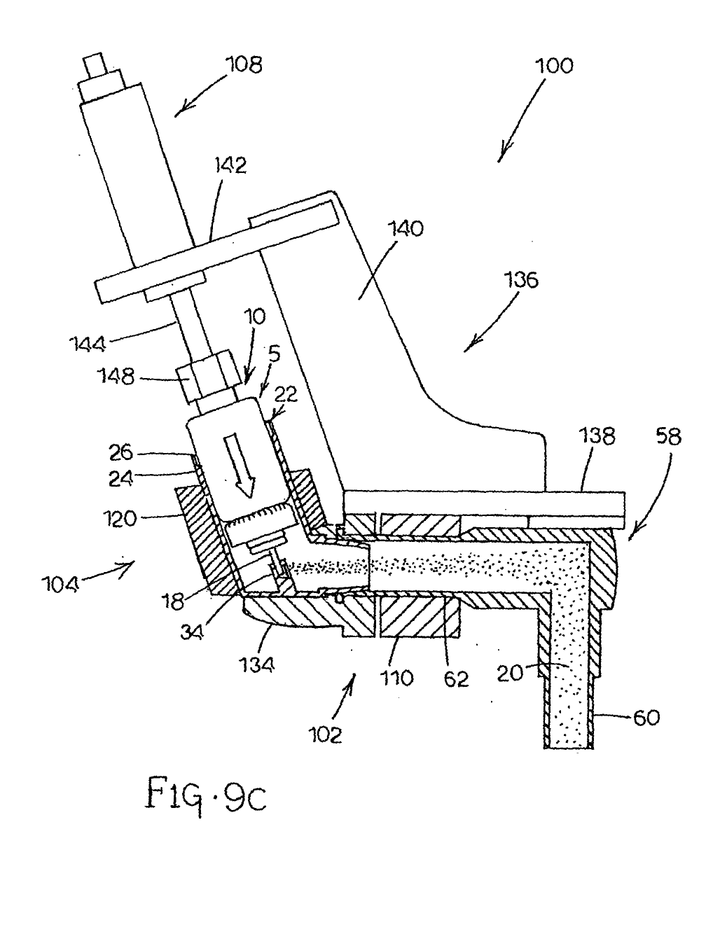

Referring to FIG. 9A, actuation assembly 108 is supported in a fixed orientation with respect to first fixture 102 by means of a mounting assembly or truss, generally designated 136. Truss 136 includes a support base 138 affixed to first clamp 110 of first fixture 102, an arm 140 supported by support base 138, and a platform 142 connecting actuation assembly 108 to arm 140. Actuation assembly 108 includes a linearly reciprocative member 144 such as a plunger or rod and an axial force measuring transducer 148 such as a load cell located in proximal association with a distal end or head 146 of linearly reciprocative member 144. As an alternative, reciprocative member 144 could comprise a rotary actuator with a linearly reciprocative member such as an arm.

Actuation assembly 108 can be any device adapted for converting an input of supplied power into a forceful axial displacement of reciprocative member 144. Additionally, in another embodiment, it should be appreciated that actuation may be carried out manually if so desired. By way of example, actuation assembly 108 can include a pneumatic cylinder, a hydraulic cylinder, linear motor and gear assembly, or a solenoid. In the case of a non-pneumatic actuation method such as use of a motor and gear assembly, transducer 148 could be eliminated in favor of measuring the current draw on the motor and correlating such measurement to a measurement of force. At the present time, it is preferred that actuation assembly 108 comprise a pneumatic cylinder, such that reciprocative member 144 includes a piston as understood by persons skilled in the art. As described more fully below, actuation assembly 108 causes reciprocative member 144 to extend or translate axially and bear down on canister 10 of delivery device 22, thereby actuating MDI unit 5 In an automated fashion. When testing apparatus 100 is in its aligned position as shown in FIGS. 8 and 9A, truss 136 ensures that reciprocative member 144 is axially aligned with canister 10, valve stem 18, and nozzle assembly 34. This arrangement in turn ensures that actuation assembly 108 operates properly and thus that MDI unit 5 is actuated properly. Preferably, actuation assembly 108 is of the double-acting type, in which a power input is required to both extend and retract reciprocative member 144 so that all movements of reciprocative member 144 can be controlled. Alternatively, a single-action actuation unit could be provided in which a spring is utilized to retract reciprocative member 144 after application of power thereto.

In an alternative embodiment, actuation assembly 108 may be mounted to second fixture 104 instead of first fixture 102. In another alternative embodiment, actuation assembly 108 is neither mounted to first fixture 102 nor to second fixture 104, but may instead be mounted to a stationary member or frame in a manner such that reciprocative member 144 of actuation assembly 108 is properly situated directly above canister 10 in axial alignment with canister 10.

The operation of actuation assembly 108 will now be described with reference to FIGS. 9A-9C. Actuation generally occurs when reciprocative member 144 of actuation assembly 108 thrusts canister 10 into canister cavity 28 of delivery device 22. More specifically, before initiation of the actuation stroke, reciprocative member 144 is located in a retracted position above canister 10, as shown in FIG. 9A. Once the ?actuation stroke is initiated, reciprocative member 144 is forced in a downward direction toward exposed end 12 of canister 10 as shown by the arrow in FIG. 9B. As also shown in FIG. 9B, reciprocative member 144 continues in the downward direction during the actuation stroke and contacts exposed end 12 of canister 10. Following contact, actuation occurs in which reciprocative member 144 thrusts, with a downward force, canister 10 into canister cavity 28 of delivery device 22, as shown in FIG. 9C. Actuation occurs upon activation of valve stem 18 of valve assembly 16, thus releasing a metered dose of atomized medicament 20 from canister 10, through mouthpiece 30, through throat 58, and into cascade impactor 42 (see FIG. 4). Referring back to FIG. 9A, after actuation, reciprocative member 144 is forced in an upward direction back to the initial, retracted position above canister 10.

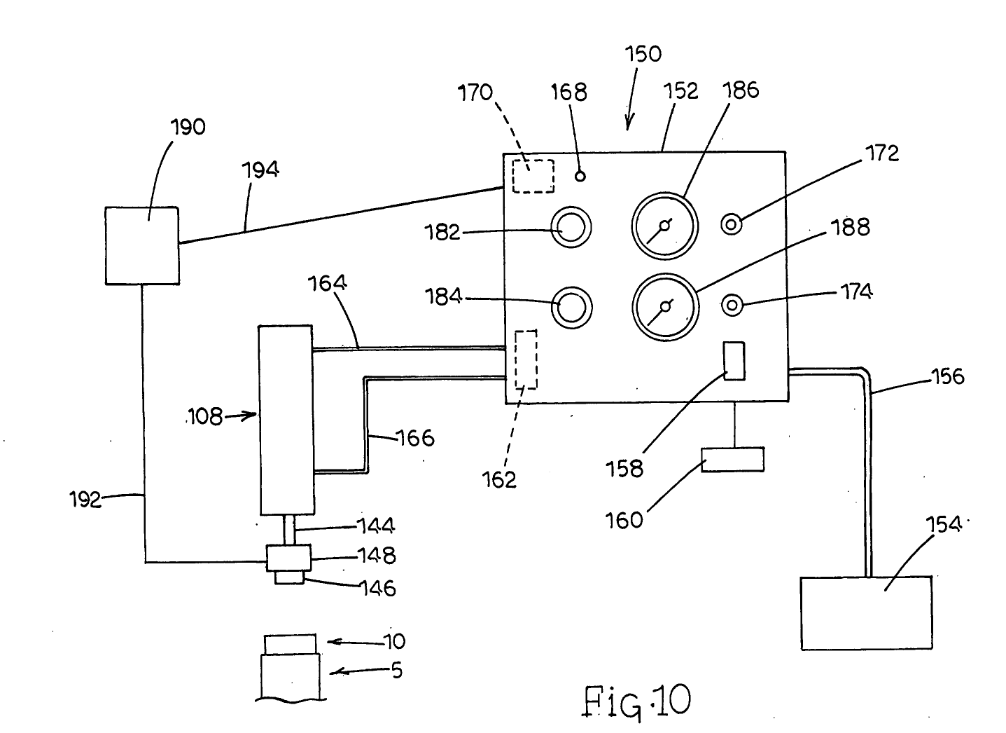

Referring now to FIG. 10, a control unit or system, generally designated 150, is provided to interface with testing apparatus 100. Preferably, control system 150 functions to control the operation of actuation assembly 108, including the extension and subsequent retraction of its reciprocative member 144. More specifically, it is preferable that control system 150 enable an operator thereof to initiate an extension/retraction sequence as well as to control the amount of power supplied to actuation assembly 108 during both the extension and retraction strokes, the respective flow rates of power supplied to actuation assembly 108 during both the extension and retraction strokes, and the duration of actuation (i.e., the time during which reciprocative member 144 is extended). These control parameters have been observed to affect the performance of metered dose delivery devices such as MDI unit 5, as well as the fluid dynamics and other characteristics of the particulate stream issuing therefrom. It is further preferable that control system 150 include a means such as an electronic circuit or device for receiving, conditioning, storing, processing and/or displaying the signals generated by and transmitted from axial force measuring transducer 148 of actuation assembly 108 during actuation of MDI unit 5.

Accordingly, FIG. 10 schematically illustrates one example of a suitable control system 150 that can be provided in the case where actuation assembly 108 comprises a double-acting pneumatic cylinder and a fluid such as air is utilized as the power medium. Control system 150 generally includes a control module 152 and an electronic circuit or device 190. A power supply source 154, such as an air compressor or a tank containing pressurized air, supplies pressurized air to control module 152 over an air supply line 156. A power switch 158 is provided to control the supply of electrical power from an electrical power source 160 to any component of control system 150 requiring electrical power for its operation.

A solenoid-activated air valve or manifold assembly 162 is provided to control the flow of pressurized air to actuation assembly 108. In the case of the preferable double-action embodiment of actuation assembly 108, two air lines 164 and 166, respectively, fluidly interconnect air valve assembly 162 and actuation assembly 108 to supply a sufficient amount of air power to initiate the respective extending and retracting strokes executed by reciprocative member 144. Air valve assembly 162 is activated by manipulation of a start button 168, which initiates the extension and retraction sequence. This sequence is controlled by a timer relay unit 170. The operator can use timer relay unit 170 to set the period of time during which air power is supplied over air line 164 to activate the extending stroke of reciprocative member 144. When this preset time expires, timer relay unit 170 interacts with air valve assembly 162 to permit air power to be supplied over air line 166 to cause reciprocative member 144 to retract.

Control system 150 also includes flow regulators to control the respective flow rates of air supplied over air lines 164 and 166. The operator can adjust these flow rates using flow rate adjustment knobs 172 and 174, respectively. In addition, pressure regulators are provided to control the respective fluid pressures of the air streams supplied over air lines 164 and 166. The operator can adjust these fluid pressures using pressure adjustment knobs 182 and 184, respectively. Pressure gauges 186 and 188, respectively, are provided for indicating the pressure levels in air lines 164 and 166. It will be understood to persons skilled in the art that additional fluid conduits and/or components (not specifically shown) are provided in control module 152 as necessary to render a complete pneumatic circuit that enables the various components of control system 150 to successfully operate and interact with associated components.

As described above, control system 150 further includes electronic circuit or device 190. Electronic circuit or device 190 communicates with axial force measuring transducer 148 of actuation assembly 108 over an electrical cable 192. Electronic device 190 functions to receive the signal or signals generated by transducer 148 proportional to the amount of axial force imparted to canister 10 by actuation assembly 108. Electronic device 190, as schematically depicted in FIG. 10, can also comprise a read-out component for providing an human-readable display of the force exerted by actuation assembly 108, a memory component for storing one or more signals received from transducer 148, a printer component for providing an archival copy of data, and signal conditioning circuitry such as an A/D converter and an amplifier. As understood by persons skilled in the art, electronic device 190 can thus represent an IC chip, a memory register, a display device, a computer terminal, a PC unit, as well as equivalents of such components, other related I/O peripheral devices, or combinations of two or more of such components. Electronic device 190 is either disposed remotely in relation to control module 152 or is integrated with control module 152. An electrical line 194 shown in FIG. 10 is intended to represent a power supply line to electronic device 190 and/or a data communication line.

It can thus be seen that control system 150 can be utilized to test MDI unit 5 by varying the axial force imparted to canister 10 during actuation thereof and the period of time during which this force is imparted. Over the course of one or more testing runs, the force data obtained from transducer 148, as well as the time of actuation set by the operator, can be correlated and compared with the particle characterization data obtained from cascade impactor 42. In this manner, optimal conditions for operation of MDI unit 5 can be investigated. Therefore, the test results obtained from the use of testing apparatus 100 in conjunction with cascade impactor 42 can be utilized to improve upon the designs of medicament delivery devices such as MDI unit 5, to evaluate different compositions and formulations of the material delivered by such device, and to modify the instructions for use of such devices provided to patients.

It is also within the scope of the invention to take advantage of the alignment provided by alignment mechanism 106 and mounting assembly 136 in order to improve procedures in which MDI unit 5 is actuated manually. That is, the invention encompasses methods in which reproducible data is obtained by using alignment mechanism 106 and mounting assembly 136 to maintain alignment integrity, but without requiring the automation provided by actuation assembly 108 and control system 150.

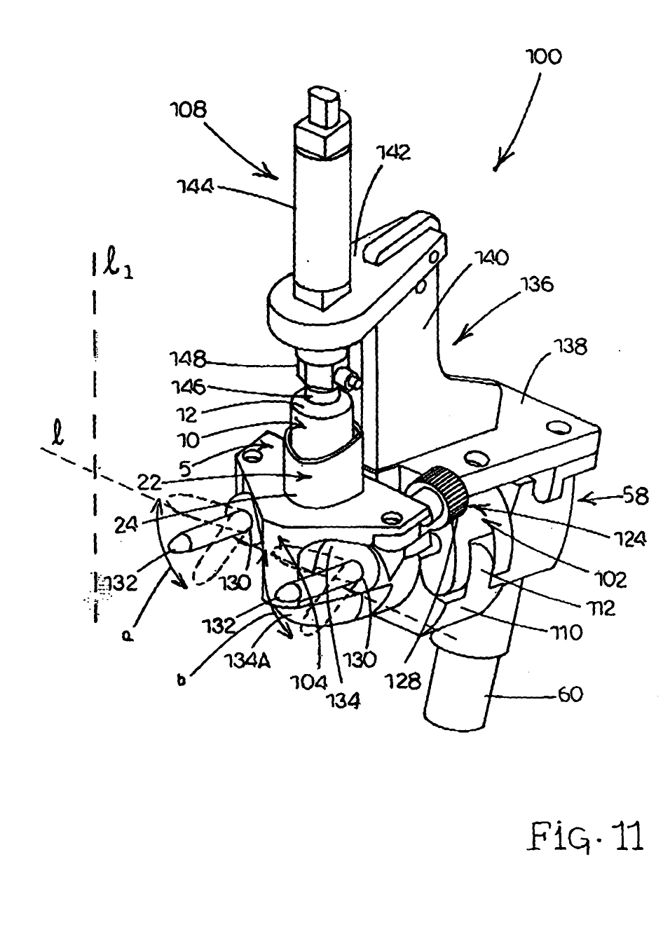

FIG. 11 illustrates another embodiment of an assembled testing apparatus in accordance with the present invention. Such an embodiment is similar to the embodiment depicted by FIG. 8. In the embodiment of FIG. 11 however, and as shown therein, alignment posts 132 may be positioned at various angles (referred to by the letters a and b) relative to a horizontal reference line, denoted by I). More specifically, alignment posts 132 may be at various locations above and below the horizontal line I as shown by the arrows in FIG. 11. Although not intending to be bound by theory, it is believed that more consistent testing results are capable of being obtained when the alignment posts are positioned at these locations since the plume spray emanating from the canister can better simulate being targeted toward a patient's throat. As a result, one can adjust the positioning of alignment bores 130 as deemed appropriate. Although alignment bores 132 can be positioned at angles so as to be present at various locations along a vertical reference line (denoted as I₁) perpendicular to the horizontal reference line I, it should be appreciated that the bores 132 may exhibit motion such that the bores are outside of vertical line I₁.

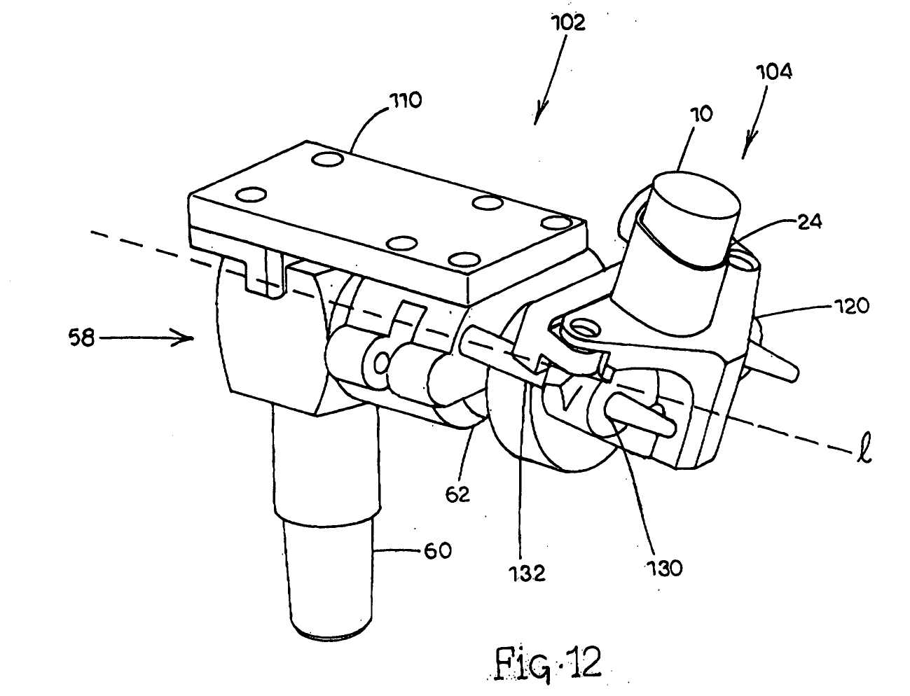

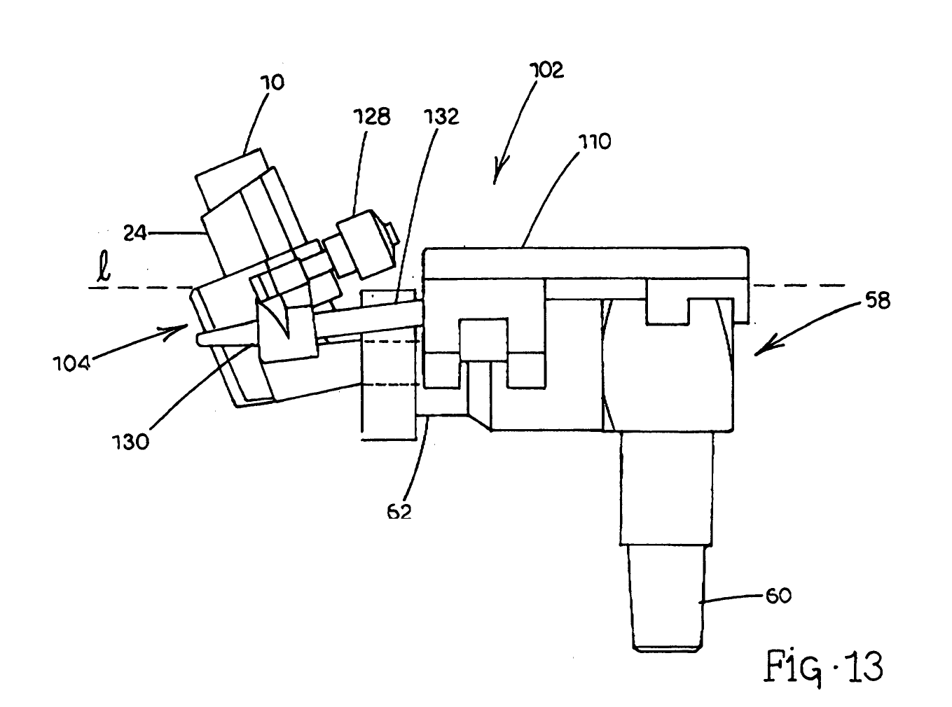

FIG. 12 illustrates a perspective view of an assembled testing apparatus according to the present invention. As shown therein, alignment bores 132 are positioned at an angle below horizontal reference line I. A similar configuration is illustrated by the cross-sectional view of the apparatus set forth in FIG. 13.

Examples of medicaments that can be tested using testing apparatus 100 and methods according to the present invention include, but are not limited to, analgesics, e.g., codeine, dihydromorphine, ergotamine, fentanyl, or morphine; anginal preparations, e.g., diltiazem; antiallergics, e.g., cromoglycate (e.g., as the sodium salt), ketotifen, or nedocromil (e.g., as the sodium salt); antiinfectives, e.g., cephalosporins, penicillins, streptomycin, sulphonamides, tetracyclines, or pentamidine; antihistamines, e.g., methapyrilene; antiinflammatories, e.g., beclomethasone (e.g., as the dipropionate ester), budesonide, ciclesonide, fluticasone (e.g., as the propionate ester), flunisolide, mometasone (e.g., as the furoate ester), rofleponide, tipredane, or triamcinolone (e.g., as the acetonide), 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., salbutamol, salmeterol (e.g., as xinafoate), ephedrine, adrenaline, fenoterol (e.g., as hydrobromide), formoterol (e.g., as fumarate), isoprenaline, metaproterenol, phenylephrine, pirbuterol (e.g., as acetate), phenylpropanolamine, reproterol (e.g., as hydrochloride), rimiterol, terbutaline (e.g., as sulphate), isoetharine, tulbuterol, orciprenaline, (-)-4-amino-3,5-dichloro-α-[[[6-[2-(2-pyridinyl)ethoxy]hexyl]amino]methyl] benzenemethanol, albuterol (e.g., as free base or sulphate), 4-hydroxy-7-[2-[[2-[[3-(2-phenylethoxy)propyl]sulfonyl]ethyl]amino]ethyl-2(3H)-benxothiazolone; adenosine 2a agonists, e.g. (2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-dioe (e.g., as maleate); α₄ integrin inhibitors, e.g. (2S)-3-[4-({[4-(aminocarbonyl)-1-piperidinyl]carbonyl}oxy)phenyl]-2-[((2S)-4-methyl-2-{[2-(2-methylphenoxy)acetyl]amino}pentanoyl)amino]propanoic acid (e.g., as free acid or potassium salt)]; diuretics, e.g., amiloride; anticholinergics, e.g., ipratropium (e.g., as bromide), tiotropium, atropine, or oxitropium; hormones, e.g., cortisone, hydrocortisone, or prednisolone; xanthines, e.g., aminophylline, choline theophyllinate, lysine theophyllinate, or theophylline; therapeutic proteins and peptides, e.g., insulin, or glucagons; and vaccines, diagnostics, and gene therapies.

It will be clear to a person skilled in the art that, where appropriate, the medicaments may be used in the form of salts (e.g. as alkali metal or amine salts or as acid addition salts), esters (e.g. lower alkyl esters), or solvates (e.g. hydrates) to optimize the activity and/or stability of the medicament and/or to minimize the solubility of the medicament in the propellant liquid.

Preferred medicaments are selected from albuterol, salmeterol, fluticasone propionate, ipratroprium and beclomethasone dipropionate and salts or solvates thereof, e.g., the sulphate of albuterol and the xinafoate of salmeterol and the bromide of ipratroprium.

Medicaments can also be delivered in combinations. Preferred formulations containing combinations of active ingredients contain salbutamol (e.g., as the free base or the sulphate salt) or salmeterol (e.g., as the xinafoate salt) or formoterol (e.g., as the fumarate salt) in combination with an anti-inflammatory steroid such as a beclomethasone ester (e.g., the dipropionate) or a fluticasone ester (e.g., the propionate) or budesonide. A particularly preferred combination is a combination of fluticasone propionate and salmeterol, or a salt thereof (particularly the xinafoate salt). A further combination of particular interest is budesonide and formoterol (e.g., as the fumarate salt).

Suitable propellants for use as a suspending medium in a sealed canister such as canister 10, and for oral inhalation by a patient include, but are not limited to, low boiling fluorocarbons, such as dichlorodifluoromethane, tetrachlorofluoroethane, 1,1,1,2-tetrafluoroethane (commonly known as “propellant 134a” or “P134a”), and 1,1,1,2,3,3,3-heptafluoropropane (commonly known as “propellant 227” or “P227”).

It will be understood that various details of the invention may be changed without departing from the scope of the invention. Furthermore, the foregoing description is for the purpose of illustration only, and not for the purpose of limitation—the invention being defined by the claims.

1. A testing apparatus for an atomized material delivery device, the testing apparatus comprising:
(a) a first fixture including a first clamp, the first clamp adjustable between an open position and a closed clamping position;
(b) a second fixture including a second clamp, the second clamp adjustable between an open position and a closed clamping position;
(c) an alignment mechanism removably coupling the first fixture to the second fixture at an aligned position of the testing apparatus; and
(d) an actuation assembly mounted to the first fixture and including a reciprocative member.

2. The testing apparatus according to claim 1, wherein the first clamp is adjustable about a first hinge between the open position and the closed clamping position of the first clamp.

3. The testing apparatus according to claim 1, wherein the first clamp includes a first locking mechanism.

4. The testing apparatus according to claim 3, wherein the first locking mechanism is pivotable about a first pivot pin disposed within the first clamp between a disengaged position and an engaged position.

5. The testing apparatus according to claim 3, wherein the first locking mechanism includes a rotatable first locking element engageable with the first clamp between a loosened position and a tightened position.

6. The testing apparatus according to claim 1, wherein the first clamp removably engages a throat.

7. The testing apparatus according to claim 1, wherein the first clamp removably engages an atomized material delivery device.

8. The testing apparatus according to claim 7, wherein the atomized material delivery device is a metered dose inhaler unit.

9. The testing apparatus according to claim 1, wherein the second clamp is adjustable about a second hinge between the open position and the closed clamping position of the second clamp.

10. The testing apparatus according to claim 1, wherein the second clamp includes a second locking mechanism.

11. The testing apparatus according to claim 10, wherein the second locking mechanism is pivotable about a second pivot pin disposed within the second clamp between a disengaged position and an engaged position.

12. The testing apparatus according to claim 10, wherein the second locking mechanism includes a rotatable second locking element engageable with the second clamp between a loosened position and a tightened position.

13. The testing apparatus according to claim 1, wherein the second clamp removably engages an atomized material delivery device.

14. The testing apparatus according to claim 13, wherein the atomized material delivery device is a metered dose inhaler unit.

15. The testing apparatus according to claim 1, wherein the second clamp removably engages a throat.

16. The testing apparatus according to claim 1, wherein the alignment mechanism includes an alignment post protruding from the first clamp and an alignment bore formed within the second clamp.

17. The testing apparatus according to claim 16, wherein the alignment post is engagingly inserted into the alignment bore at an aligned position of the testing apparatus relative to a horizontal line.

18. The testing apparatus according to claim 17, wherein the alignment post is engagingly inserted into the alignment bore in the horizontal line.

19. The testing apparatus according to claim 17, wherein the alignment post is engagingly inserted into the alignment bore at an angle relative to the horizontal line.

20. The testing apparatus according to claim 1, wherein the alignment mechanism includes an alignment post protruding from the second clamp and an alignment bore formed within the first clamp.

21. The testing apparatus according to claim 20, wherein the alignment post is engagingly inserted into the alignment bore at an aligned position of the testing apparatus relative to a horizontal line.

22. The testing apparatus according to claim 21, wherein the alignment post is engagingly inserted into the alignment bore in the horizontal line.

23. The testing apparatus according to claim 21, wherein the alignment post is engagingly inserted into the alignment bore at an angle relative to the horizontal line.

24. The testing apparatus according to claim 1 wherein, at the aligned position of the testing apparatus, a throat is in fluid communication with an atomized material delivery device.

25. The testing apparatus according to claim 24, wherein the throat is mounted to the first fixture at the closed clamping position of the first clamp and the atomized material delivery device is mounted to the second fixture at the closed clamping position of the second clamp.

26. The testing apparatus according to claim 24 wherein the reciprocative member of the actuation assembly is in axial alignment with the atomized material delivery device.

27. The testing apparatus according to claim 1, wherein the actuation assembly includes a pneumatic cylinder disposed in operative communication with the reciprocative member.

28. The testing apparatus according to claim 1, wherein the actuation assembly includes a stepper motor disposed in operative communication with the reciprocative member.

29. The testing apparatus according to claim 1, wherein the actuation assembly includes a solenoid in operative communication with the reciprocative member.

30. The testing apparatus according to claim 1, wherein the actuation assembly includes an axial force measuring transducer.

31. The testing apparatus according to claim 30 comprising a data-receiving device electrically communicating with the axial force measuring transducer.

32. The testing apparatus according to claim 1 wherein the actuation assembly includes a displacement measuring transducer.

33. The testing apparatus according to claim 1 comprising a power supply source communicating with the actuation assembly.

34. The testing apparatus according to claim 33, wherein the power supply source is a pressurized air source and the actuation assembly includes a pneumatic cylinder.

35. The testing apparatus according to claim 33, wherein the power supply source is an electrical energy source and the actuation assembly includes a solenoid.

36. The testing apparatus according to claim 1 comprising a control module for controlling the supply of power to the actuation assembly.

37. The testing apparatus according to claim 36 wherein the control module includes a timer device for controlling the period of time over which the reciprocative member is extended by the actuation assembly.

38. A medicament tested using the apparatus according to claim 1, wherein the -medicament includes a drug component selected from the group consisting of analgesics, anginal preparations, anti-allergenics, anti-infectives, antihistamines, anti-inflammatories, anti-tussives, bronchodilators, adenosine 2a agonists, α₄ integrin inhibitors, diuretics, anticholinergics, hormones, xanthines, therapeutic proteins, therapeutic peptides, vaccines, diagnostics, gene therapies, and salts, esters and solvates thereof, and combinations thereof.

39. An apparatus for aligning an atomized material delivery device such as a metered dose delivery unit with a particle characterization device, the apparatus comprising:
(a) an inlet conduit;
(b) a first fixture removably secured to the inlet conduit;
(c) an atomized material delivery device having an outlet conduit;
(d) a second fixture removably secured to the delivery device; and
(e) an alignment mechanism intercoupling the first fixture to the second fixture at an aligned position of the apparatus, wherein the inlet conduit fluidly communicates with the outlet conduit at the aligned position of the apparatus.

40. The apparatus according to claim 39 comprising a particle characterization component, the particle characterization component including the inlet conduit.

41. The apparatus according to claim 40 wherein the particle characterization component is a throat.

42. The apparatus according to claim 39 comprising an adapter element interconnecting the inlet conduit with the outlet conduit at the aligned position of the apparatus.

43. The apparatus according to claim 39 wherein the alignment mechanism includes a guide member supported by the first fixture, wherein the guide member is inserted into a bore of the second fixture at the aligned position of the apparatus.

44. The apparatus according to claim 39 comprising an actuation mechanism including a reciprocative member axially aligned with the delivery device at the aligned position of the apparatus.

45. The apparatus according to claim 44 comprising an actuation control module disposed in operative communication with the actuation mechanism.

46. The apparatus according to claim 44 comprising an axial force measurement transducer operatively mounted to the actuation mechanism.

47. The apparatus according to claim 39, wherein the actuation mechanism includes a stepper motor.

48. A medicament tested using the apparatus according to claim 39, wherein the medicament includes a drug component selected from the group consisting of analgesics, anginal preparations, anti-allergenics, anti-infectives, antihistamines, anti-inflammatories, anti-tussives, bronchodilators, adenosine 2a agonists, α₄ integrin inhibitors, diuretics, anticholinergics, hormones, xanthines, therapeutic proteins, therapeutic peptides, vaccines, diagnostics, gene therapies, and salts, esters and solvates thereof, and combinations thereof.

49. A testing apparatus for providing alignment and automated actuation during particle analysis of an atomized medicament emitted from a delivery device and conducted into a particle characterization device, the testing apparatus comprising:
(a) a particle characterization device including an inlet conduit;
(b) a first fixture including a first clamp in removable clamping engagement with the inlet conduit;
(c) a metered dose inhaler unit including a delivery device and a particle-containing canister operatively loaded in the delivery device;
(d) a second fixture including a second clamp in removable clamping engagement with the delivery device of the metered dose inhaler unit;
(e) an alignment mechanism including an alignment post protruding from the first clamp and an alignment bore formed within the second clamp, the alignment post adapted to be engagingly inserted into the alignment bore at an aligned position of the testing apparatus relative to a horizontal line so as to provide fluid communication between the inlet conduit and the delivery device of the metered dose inhaler unit; and
(f) an actuation assembly mounted to the first fixture and including a reciprocative member positioned in operative alignment with the canister, the reciprocative member adapted to thrust the canister into the delivery device to cause the delivery device to release a metered dose of particles from the canister.

50. The testing apparatus according to claim 49, wherein the alignment post is engagingly inserted into the alignment bore in the horizontal line.

51. The testing apparatus according to claim 49, wherein the alignment post is engagingly inserted into the alignment bore at an angle relative to the horizontal line.

52. The testing apparatus according to claim 49, wherein the reciprocative member includes an axial force measuring transducer.

53. The testing apparatus according to claim 49 comprising a control system for controlling the operation of the actuation assembly.

54. A medicament tested using the apparatus according to claim 49, wherein the medicament includes a drug component selected from the group consisting of analgesics, anginal preparations, anti-allergenics, anti-infectives, antihistamines, anti-inflammatories, anti-tussives, bronchodilators, adenosine 2a agonists, α₄ integrin inhibitors, diuretics, anticholinergics, hormones, xanthines, therapeutic proteins, therapeutic peptides, vaccines, diagnostics, gene therapies, and salts, esters and solvates thereof, and combinations thereof.

55. A method for testing the aerodynamic particle size of an atomized medicament comprising:
(a) engaging an inlet portion of a particle characterization device with a first fixture;
(b) operatively loading a canister containing particles into an atomized material delivery device;
(c) engaging the delivery device with a second fixture;
(d) effecting an aligned position of the testing apparatus by removably coupling the first fixture to the second fixture so as to provide fluid communication between the inlet conduit and the delivery device; and
(e) actuating the release of particles from the canister.

56. The method according to claim 55, wherein the step of effecting the aligned position of the testing apparatus includes aligning an alignment post protruding from the first fixture with an alignment bore formed within the second fixture, and inserting the alignment post into the alignment bore.

57. The method according to claim 55, wherein the step of actuating the release of particles from the canister includes manually applying a force to the canister.

58. The method according to claim 55, wherein the step of actuating the release of particles from the canister includes causing a linearly translatable member to impact the canister with an axially oriented force.

59. The method according to claim 58 comprising the step of measuring a magnitude of an axial force imparted to the canister during the actuating step.
